(19) United States
(12) Patent Application Publication
(10) Pub. No.: US 2018/0099154 A1
(43) Pub. Date: Apr. 12, 2018

(54) MEDICAL APPARATUS COMPRISING A HADRON THERAPY DEVICE, A MRI, AND A PROMPT-GAMMA SYSTEM

(71) Applicant: Ion Beam Applications S.A.

(72) Inventor: Damien PRIEELS, Court-Saint-Etienne (BE)

(73) Assignee: Ion Beam Applications S.A.

(21) Appl. No.: 15/726,836

(22) Filed: Oct. 6, 2017

(30) Foreign Application Priority Data
Oct. 7, 2016 (EP) 16192796.7

(51) Int. Cl. A61N 5/10

(52) U.S. Cl. CPC A61N 5/1039; A61N 5/1049; A61N 2005/1058; A61N 2005/1055; A61N 2005/1052; A61N 2005/1087

(57) ABSTRACT

The present disclosure relates to a medical apparatus. In one implementation, the medical apparatus includes a hadron therapy device adapted for directing a hadron beam having an initial beam energy along a beam path to a target spot located inside a subject of interest; an MRI for acquiring a magnetic resonance (MR) image within an imaging volume having the target spot; a prompt-γ system adapted for acquiring a signal generated by the hadron beam; and a controller configured for computing an actual position of the Bragg peak of the hadron beam, based on the signal acquired by the PG system, and locating the actual position of the Bragg peak on the MR image.

### CROSS-REFERENCE TO RELATED APPLICATIONS

This application claims the benefit of priority to European Application No. 16192796.7, filed Oct. 7, 2016, the contents of which are incorporated herein by reference.

### TECHNICAL FIELD

The present disclosure relates to a medical apparatus comprising a charged hadron therapy device, a magnetic resonance imaging device, and a prompt-gamma (prompt-γ) system. The present disclosure further relates to methods for checking a treatment plan.

### BACKGROUND

Hadron therapy (for example, proton therapy) for treating a patient may provide several advantages over conventional radiotherapy. These advantages are generally due to the physical nature of hadrons. For example, a photon beam in conventional radiotherapy releases its energy according to a decreasing exponential curve as a function of the distance of tissue traversed by the photon beam. By contrast, and as illustrated in the example of FIG. 2A, a hadron beam first releases a small fraction of its energy as it penetrates tissues 41-43, forming a plateau, then, as the hadron path is prolonged, releases energy locally following a steep increase to a peak and a fall-off at the end of the range of the beam. The peak is called a Bragg peak and corresponds to the maximum of the Bragg curve illustrated in the example of FIG. 2C. Consequently, a hadron beam may deliver a high dose of hadrons at a precise location within a target tissue 40 and may therefore preserve the surrounding healthy tissues 41-44. As illustrated in the example of FIG. 2A, if the position, BP0, of the Bragg peak of a hadron beam is offset relative to the target tissues 40, high doses of hadrons may be delivered to adjacent tissues 43, 44, which are healthy (as illustrated with solid line, E0, and dashed line, E0d, of the curves of energy loss, Eₗₒₛₛ, with respect to the distance, Xh, travelled by the hadron beam within tissues and measured along the beam path, Xp, in the example of FIG. 2A). For this reason, the determination of the relative position of the Bragg peak with respect to the position of the target tissue is often crucial to properly implement hadron therapy to a patient.

In practice, hadron therapy usually requires the establishment of a treatment plan before any treatment can start. During this treatment plan, a computer tomography scan (CT scan) of the patient and target tissues is generally performed. The CT scan may be used to characterize the target tissue 40 and the surrounding tissues 41-43 to be traversed by a treatment hadron beam 1h for the treatment of a patient. The characterization may yield a 3D representation of the volume comprising the target tissue, and a treatment plan system may determine a range-dose calculated based on the nature of the tissues 41-43 traversed by the hadron beam.

This characterization may allow computation of a water equivalent path length (WEPL), which may be used for determining the initial energy, Ek, of the treatment hadron beam required for delivering a prescribed dose of hadrons to a target spot 40s, wherein k=0 or 1, depending on the stage when said initial energy was determined. The example of FIG. 2C illustrates the conversion of the physical distances travelled by a hadron beam traversing different tissues into corresponding WEPLs. The WEPL of a hadron beam travelling a given distance through a given tissue is the equivalent distance said hadron beam would travel in water. As illustrated in the example of FIG. 2C if, as is often the case, healthy tissues 41-43 of different natures and thicknesses separate a target tissue from the outer surface of the skin of a patient, the WEPL of a target spot may be calculated taking into account the water corresponding path lengths of each tissue in series until the target spot is reached. With a value of the equivalent path length of a hadron beam traveling in water, the initial energy, Ek, required for positioning the Bragg peak at the WEPL of the target spot may be computed and correspond to the initial energy, Ek, required for positioning the Bragg peak at the target spot within the target tissue.

The treatment plan may then be executed during a treatment phase including one or more treatment sessions during which doses of hadrons are deposited onto the target tissue. The position of the Bragg peak of a hadron beam with respect to the target spots of a target tissue, however, may suffer of a number of uncertainties including:
the variations of the patient position, on the one hand, during a hadron therapy session and, on the other hand, between the establishment of the treatment plan and the hadron therapy session;
the variations of the size and/or of the position of the target tissue (see, for example, FIG. 2B) and/or of the healthy tissues 41-43 positioned upstream from the target tissue with respect to the hadron beam; and
the range calculation from CT scans being limited by the quality of the CT images. Another limitation is linked to the fact that CT scans use the attenuation of X-rays that have to be converted in hadron attenuation, which may depend on the chemical composition of the tissues traversed.

The uncertainty on the position of the patient and, in particular, of the target tissue may be critical. Even with an accurate characterization by CT scan, the actual position of a target tissue during a treatment session may remain difficult to ascertain for the following reasons:
(A) first, during an irradiation session, the position of a target tissue may change because of anatomical processes such as breathing, digestion, or heartbeats of the patient. Anatomical processes may also cause gases or fluids appearing or disappearing from the beam path, Xp, of a hadron beam.
(B) second, treatment plans are generally determined several days or weeks before a hadron treatment session starts and treatment of a patient may take several weeks distributed over several treatment sessions. During this time period, the patient may lose or gain weight, therefore modifying, sometimes significantly, the volume of tissues such as fats and muscles.

Accordingly, the size of the target tissue may change (e.g., a tumour may have grown, receded, or changed position or geometry). The example of FIG. 2B shows an example of evolution of the size and position of a target tissue 40 between the time, t0, of the establishment of the treatment plan and the times, t0+Δt1, t0+Δt2, t1=t0+Δt3, of treatment sessions. The treatment plan and last treatment session may be separated by several days or weeks. The treatment plan established at time, t0, may therefore comprise irradiation of a target spot 40si,j (black spot in the example of FIG. 2B), which belonged to the target tissue 40p at said time, t0. Because the target tissue 40p may have moved or changed shape during the time period, Δt3, said target spot 40si,j may not belong to the target tissue 40 anymore at the time, t0+Δt3, of the treatment session and may be located in a healthy tissue instead. Consequently, irradiating said target spot may hit and possibly harm healthy tissues 43 instead of target tissues 40.

The use of a magnetic resonance imaging device (MRI) coupled to a hadron therapy device has been proposed for identifying any variation of the size and/or the position of a target tissue. For example, U.S. Pat. No. 8,427,148 generally relates to a system comprising a hadron therapy device coupled to an MRI. Said system may acquire images of the patient during a hadron therapy session and may compare these images with CT scan images of the treatment plan. FIG. 1 illustrates an example of a flowchart of a hadron therapy session using a hadron therapy device coupled to an MRI. A treatment plan may be established including the characterization of the target tissue 40s and surrounding tissues 41-43. This step is generally performed with a CT scan analysis and allows the determination of the position, P0, and morphology of a target tissue, the best trajectories or beam paths, Xp, of hadron beams for the hadron treatment of the target tissue, and characterization of the sizes and natures of the tissues traversed by a hadron beam following said beam paths, Xp, to determine WEPLs of target spots of the said target tissue. The initial energies, Ek, of the hadron beams required for matching the corresponding positions, BP0, of the Bragg peaks of the hadron beams to the position, P0, of the target tissue may thus be calculated. This generally completes the establishment of a treatment plan.

A hadron therapy session may follow the establishment of the treatment plan. With an MRI coupled to a hadron therapy device, it may be possible to capture a magnetic resonance (MR) image of a volume, Vp, including the target tissue and surrounding tissues to be traversed by a hadron beam. The MR image may then be compared with CT scan images to assess whether any morphological differences, Δ, can be detected in the imaged tissues between the time the CT scans were performed (=t0 in the example of FIG. 2B) and the time of the hadron therapy session (t1=t0+Δt3 in the example of FIG. 2B). If no substantial difference in morphology affecting the treatment session is detected, then the hadron therapy session may proceed as planned in the treatment plan. If, on the other hand, some differences are detected that could influence the relative position of the target tissue with respect to the planned hadron beams and their respective Bragg peaks, the hadron therapy session may be interrupted and a new treatment plan established. This technique may prevent carrying out a hadron therapy session based on a treatment plan that has become obsolete, which may prevent healthy tissues from being irradiated instead of the target tissue.

The magnetic resonance (MR) images generally provide high contrast of soft tissue traversed by a hadron beam but, at the time of filing, have usually not been suitable for visualizing the hadron beam itself, let alone the position of the Bragg peak because:
MRI measures the density of hydrogen atoms in tissues but, at the time of filing, does not usually yield any identifiable information on the hadron stopping power ratio. The conversion from density of hydrogen atoms to the hadron stopping power ratio suffers from uncertainties similar to and yet generally less understood than those of the conversion from X-rays in CT scan.
Due to the different techniques used in CT scan and in MRI, the comparison between the images from CT scan and the images from MRI may suffer from uncertainties.

In conclusion, in hadron therapy, an accurate determination of the position of the Bragg peak relative to the portion of a target tissue is important because errors regarding this position may lead to the irradiation of healthy tissues rather than irradiation of target tissues. However, no satisfactory solution for determining the relative positions of the Bragg peak and target tissues is presently available. Apparatuses combining a hadron therapy device and an MRI may allow in situ acquisition of images during a treatment session, thus giving information related to the actual position of the target tissue. Said images are, however, generally insufficient for ensuring a precise determination of the position of the Bragg peak of a hadron beam and of its location relative to the target tissue. Accordingly, there remains a need for a hadron therapy device combined with an MRI that allows a better determination of the position of the Bragg peak relative to the position of a target tissue.

### SUMMARY

According to a first aspect, a medical apparatus may comprise:
(A) a hadron therapy device comprising a hadron source adapted for directing a hadron beam having an initial beam energy, E0, along a beam path to a target spot located inside a subject of interest;
(B) a magnetic resonance imaging device (MRI) for acquiring magnetic resonance (MR) image within an imaging volume, Vp, comprising the target spot;
(C) a prompt-γ system adapted for acquiring a signal generated by the hadron beam; and
(D) a controller configured for:
   computing an actual position, BP1, of the Bragg peak of said hadron beam, based on the signal acquired by the prompt-γ system; and
   locating the actual position, BP1, of the Bragg peak on an MR image of the imaging volume, Vp, acquired with the MRI along the beam path from an outer surface of the subject of interest to the target spot.

The medical apparatus may further comprise a display, and the controller may be configured for representing, on a same coordinate scale, the MR image obtained from the MRI and the position of the Bragg peak obtained from the prompt-γ system.

The controller may also be configured for comparing the actual position, BP1, of the Bragg peak and the actual position, P1, of the target spot.

In some embodiments, when the actual position, BP1, and the position, P1, of the target spot are offset by a distance greater than a given tolerance, the controller may be further configured for computing the water equivalent path lengths of each tissue m, crossed by the beam path and between the outer surface and the target spot. The computation may be based on the thickness Lm and nature of each tissue m determined on the MR image, and on the water equivalent path length corresponding to the distance between the outer surface and the target spot determined by the prompt-γ system.

The tolerance may be less than ±10 mm, e.g., ±5 mm or ±3 mm.

In some embodiments, the controller may be configured for optimising the treatment plan by correcting the value of planned initial beam energy E0 of target spot, to a corrected initial beam energy E1, suitable for matching the positions of the Bragg peak of said hadron beam with the positions of all target spots located in a same iso-energy volume, Vti.

In an alternative embodiment, the prompt-γ system may be replaced by at least one of a PET system and an ultrasound system.

The medical apparatus may further comprise a hadron radiography system and/or a support for supporting a patient in a non-supine position.

According to a second aspect, a method for locating the Bragg peak of a hadron beam having an initial beam energy, E0 and being emitted along a beam path to a target spot within a target tissue may comprise:
(A)performing a magnetic resonance (MR) imaging of an imaging volume, Vp, comprising a target spot, and acquiring an MR image;
(B) emitting, along the beam path to the target spot, the hadron beam having an initial beam energy, E0;
(C) detecting a signal generated by said hadron beam with a prompt-γ system;
(D) from said signal, determining an actual position, BP1, of the Bragg peak of said hadron beam, based on the signal acquired by the prompt-γ system;
(E) locating the actual position, BP1, of the Bragg peak on the MR image of the imaging volume, Vp, acquired with the MRI along the beam path from an outer surface of the subject of interest to the target spot.

The method may further comprise comparing the actual position, BP1, of the Bragg peak and the actual position, P1, of the target spot.

In some embodiments, when the actual position, BP1, and the actual position, P1, of the target spot are offset by a distance greater than a given tolerance, the method may further comprise computing the water equivalent path lengths of each tissue m, crossed by the beam path and between the outer surface and the target spot. The computation may be based on the thickness Lm and nature of each tissue m determined on the MR image, and on the water equivalent path length corresponding to the distance between the outer surface and the target spot determined by the prompt-γ system.

The tolerance may be less than ±10 mm, e.g., ±5 mm or ±3 mm.

In some embodiments, the method may further comprise optimising the treatment plan by correcting the value of planned initial beam energy E0 of target spot, to a corrected initial beam energy E1, suitable for matching the positions of the Bragg peak of said hadron beam with the positions of all target spots located in a same iso-energy volume, Vti.

The method may further comprise:
(A) providing a display; and
(B) representing, on a same coordinate scale, the magnetic resonance data obtained from the MRI and the actual position of the Bragg peak obtained from the prompt-γ system.

In some embodiments, performing a magnetic resonance (MR) imaging and emitting an imaging hadron beam are done in the same room.

In some embodiments, the methods according to the present disclosure may further comprise providing a medical apparatus according to the present disclosure.

### SHORT DESCRIPTION OF THE DRAWINGS

These and further aspects of the present disclosure will be explained in greater detail by way of example and with reference to the accompanying drawings in which:

FIG. 1 shows a flowchart of a hadron therapy method using a hadron therapy device coupled to a MRI.

FIG. 2A schematically shows the position of the Bragg peak of a hadron beam traversing tissues.

FIG. 2B schematically shows changes with time of the morphology and position of a target tissue that can create a discrepancy between a treatment plan and an actual required treatment.

FIG. 2C schematically shows the relationship between actual path lengths and water equivalent path lengths.

FIG. 3A schematically shows a medical apparatus comprising a hadron therapy device coupled to a MRI, according to an example embodiment of the present disclosure.

FIG. 3B schematically shows another medical apparatus comprising a hadron therapy device coupled to a MRI, according to another example embodiment of the present disclosure.

FIG. 4A schematically illustrates a nozzle mounted on a gantry for delivering a therapeutic dose of hadron, according to an example embodiment of the present disclosure.

FIG. 4B illustrates volumes of target tissue receiving a therapeutic dose of hadron from the nozzle of FIG. 4A, according to an example embodiment of the present disclosure.

FIG. 4C illustrates a dose of hadron delivered to the target tissue of FIG. 4B, according to an example embodiment of the present disclosure.

FIG. 5A schematically shows a selection of an imaging slice in an MRI, according to an example embodiment of the present disclosure.

FIG. 5B schematically shows a creation of phase gradients and frequency gradients during imaging of the slice of FIG. 5A, according to an example embodiment of the present disclosure.

FIG. 6A shows an example of an apparatus according to an example embodiment of the present disclosure, showing access of a hadron beam to a target tissue.

FIG. 6B shows another example of an apparatus according to another example embodiment of the present disclosure, showing access of a hadron beam to a target tissue.

FIG. 7 shows an example of a medical apparatus comprising a hadron therapy device, a MRI device, and a hadron radiography system, according to an example embodiment of the present disclosure.

FIG. 8 schematically illustrates an example detector for a prompt-γ system, according to an example embodiment of the present disclosure.

FIG. 9 schematically illustrates the computation of the energy of a hadron beam, according to an example embodiment of the present disclosure.

FIG. 10 shows a flowchart of an example hadron therapy method using a medical apparatus according to an example embodiment of the present disclosure.

FIG. 11 shows an example of a medical apparatus comprising a hadron therapy device, a MRI device, and a PET scan, according to an example embodiment of the present disclosure.

FIG. 12 shows an example embodiment of a medical apparatus comprising a hadron therapy device, a MRI device, and an ultrasound system, according to an example embodiment of the present disclosure.

FIG. 13 shows an example embodiment of a medical apparatus comprising a hadron radiography system, according to an example embodiment of the present disclosure.

FIG. 14 shows an example embodiment of a medical apparatus comprising a support for supporting a patient in a non supine position, according to an example embodiment of the present disclosure.

The figures are not drawn to scale. Generally, identical components are denoted by the same reference numerals in the figures.

### DETAILED DESCRIPTION

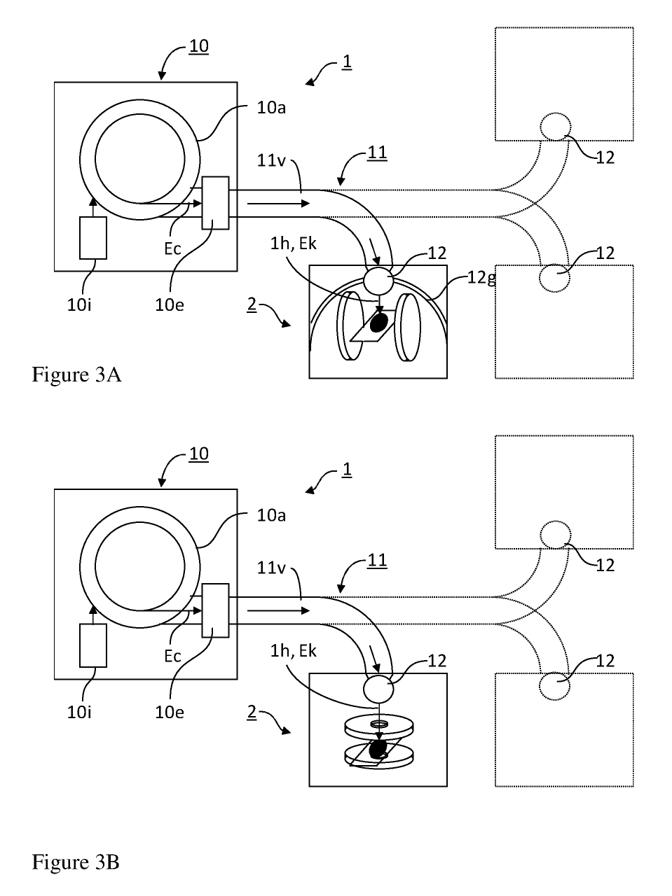

Hadron therapy is a form of external beam radiotherapy using beams 1h of energetic hadrons. FIGS. 3A, 3B, 4A, 6A, and 6B show a hadron beam 1h directed towards a target spot 40s in a target tissue 40 of a subject of interest. Target tissues 40 of a subject of interest typically include cancerous cells forming a tumour. During a hadron therapy session, a hadron beam of initial energy, Ek, with k=0 or 1, may irradiate one or more target spots within the target tissue, such as a tumour, and destroy the cancerous cells included in the irradiated target spots, reducing the size of the treated tumour by necrosis of the irradiated tissues.

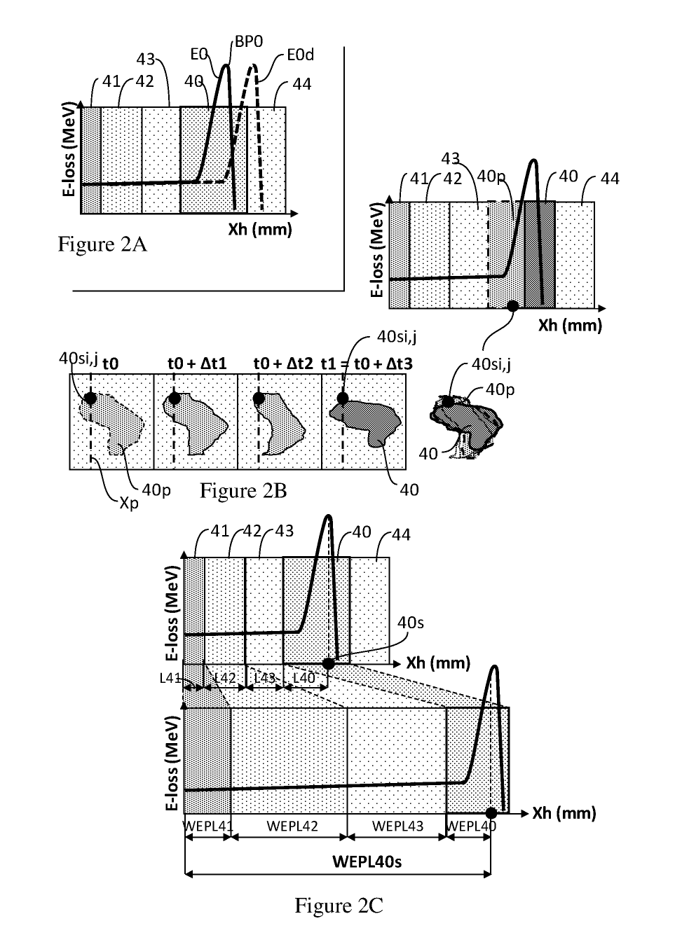

The subject of interest may comprise a plurality of materials including organic materials. For example, the subject of interest may comprise a plurality of tissues m, with m=40-44 as shown in the example of FIGS. 2A, 2B, and 2C, that may be, for example, skin, fat, muscle, bone, air, water (and/or blood), organ, tumour, or the like. For example, the target tissue 40 may be a tumour.

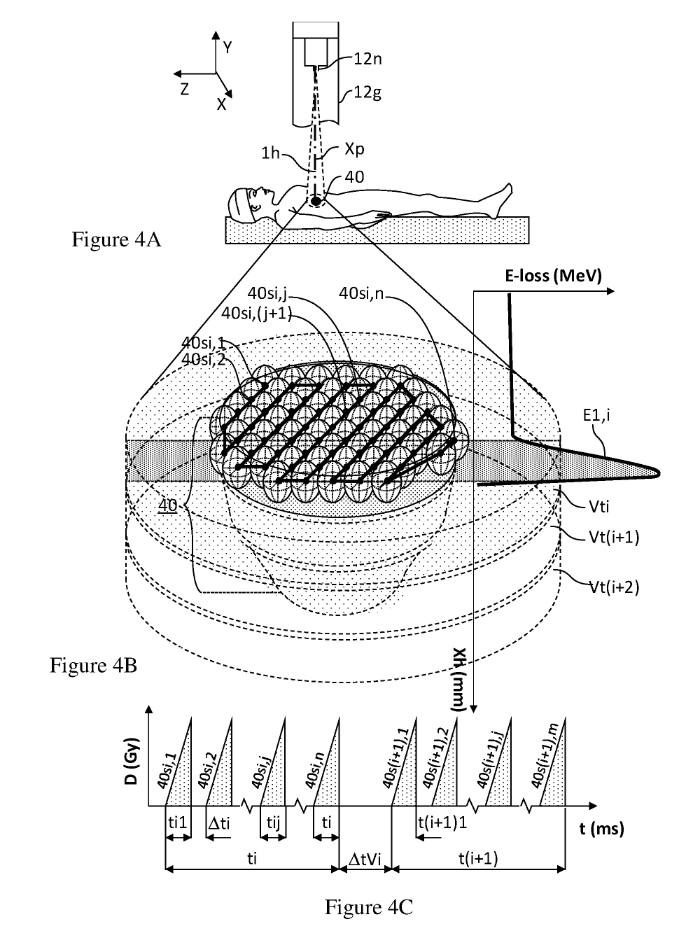

A hadron beam 1h traversing an organic body along a beam path, Xp, generally loses most of its energy at a specific distance of penetration along the beam path, Xp. As illustrated in FIGS. 2A, 2B, 2C, and 4B, said specific distance of penetration may correspond to the position of the Bragg peak, observed when plotting the energy loss per unit distance [MeVg⁻¹ cm⁻²], Eₗₒₛₛ, of a hadron beam as a function of the distance, xh, measured along the beam path, Xp. Unlike other forms of radiation therapies, a hadron beam may therefore deliver a high dose of energy at a very specific location within a target tissue corresponding to the position of the Bragg peak. The position of the Bragg peak may depend mainly on the initial energy, Ek, of the hadron beam (i.e., before traversing any tissue) and on the nature and thicknesses of the traversed tissues. The hadron dose delivered to a target spot may depend on the intensity of the hadron beam and on the time of exposure. The hadron dose may be measured in Grays (Gy), and the dose delivered during a treatment session is usually of the order of one to several Grays (Gy).

A hadron is a composite particle made of quarks held together by strong nuclear forces. Typical examples of hadrons may include protons, neutrons, pions, heavy ions, such as carbon ions, and the like. In hadron therapy, electrically charged hadrons are often used. For example, the hadron may be a proton, and the corresponding hadron therapy may be referred to as proton therapy. Accordingly, in the following description, unless otherwise indicated, any reference to a proton beam and/or proton therapy may apply to a hadron beam and/or hadron therapy in general.

A hadron therapy device 1 generally comprises a hadron source 10, a beam transport line 11, and a beam delivery system 12. Charged hadrons may be generated from an injection system 10i, and may be accelerated in a particle accelerator 10a to build up energy. Suitable accelerators may include, for example, a cyclotron, a (synchro-)cyclotron, a synchrotron, a laser accelerator, or the like. For example, a (synchro-)cyclotron may accelerate charged hadron particles from a central area of the (synchro-)cyclotron along an outward spiral path until the particles reach the desired output energy, Ec, whence they may be extracted from the (synchro-)cyclotron. Said output energy, Ec, reached by a hadron beam when extracted from the (synchro-)cyclotron is typically between 60 and 400 MeV, e.g., between 210 and 250 MeV. The output energy, Ec, may be, but is not necessarily, the initial energy, Ek, of the hadron beam used during a therapy session. For example, Ek may be equal to or lower than Ec, such that Ek≤Ec. An example of a suitable hadron therapy device may include, but is not limited to, a device described in U.S. Pat. No. 4,870,287, the entire disclosure of which is incorporated herein by reference as representative of a hadron beam therapy device used in the present disclosure.

The energy of a hadron beam extracted from a (synchro-)cyclotron may be decreased by energy selection means 10e, such as energy degraders or the like, positioned along the beam path, Xp, downstream of the (synchro-)cyclotron. Energy selection means 10e may decrease the output energy, Ec, down to any value of Ek, including down to nearly 0 MeV. As discussed supra, the position of the Bragg peak along a hadron beam path, Xp, traversing specific tissues may depend on the initial energy, Ek, of the hadron beam. By selecting the initial energy, Ek, of a hadron beam intersecting a target spot 40s located within a target tissue, the position of the Bragg peak may be controlled to correspond to the position of the target spot.

A hadron beam may also be used for characterizing properties of tissues. For example, images may be obtained with a hadron radiography system (HRS), for example, a proton radiography system (PRS). The doses of hadrons delivered to a target spot for characterization purposes, however, may be considerably lower than the doses delivered during a hadron therapy session, which, as discussed supra, may be of the order of 1 to 10 Gy. The doses of delivered hadrons of HRS for characterization purposes are typically of the order of 10⁻³ to 10⁻¹ Gy (i.e., one to four orders of magnitude lower than doses typically delivered for therapeutic treatments). These doses may have no significant therapeutic effects on a target spot. Alternatively or concurrently, a treatment hadron beam delivered to a small set of target spots in a target tissue may be used for characterization purposes. The total dose delivered for characterization purposes may be insufficient to treat a target tissue.

As illustrated in FIGS. 3A and 3B, downstream of the hadron source, a hadron beam of initial energy, Ek, may be directed to the beam delivery system 12 through a beam transport line 11. The beam transport line may comprise one or more vacuum ducts, 11v, and a plurality of magnets for controlling the direction of the hadron beam and/or for focusing the hadron beam. The beam transport line may also be adapted for distributing and/or selectively directing the hadron beam from a single hadron source 10 to a plurality of beam delivery systems for treating several patients in parallel.

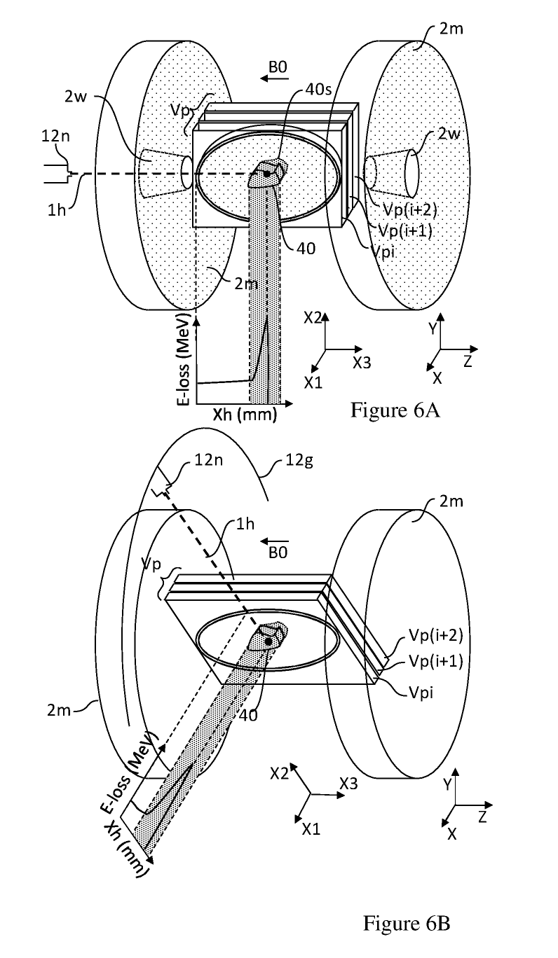

The beam delivery system 12 may further comprise a nozzle 12n for orienting a hadron beam 1h along a beam path, Xp. The nozzle may be fixed or mobile. Mobile nozzles are generally mounted on a gantry 12g, as illustrated schematically in the examples of FIGS. 4A and 6B. A gantry may be used for varying the orientation of the hadron outlet about a circle centred on an isocentre and normal to an axis, Z, which may be horizontal. In supine hadron treatment devices, the horizontal axis, Z, may be selected parallel to a patient lying on a couch (i.e., the head and feet of the patient are aligned along the horizontal axis, Z). The nozzle 12n and the isocentre define a path axis, Xn, whose angular orientation depends on the angular position of the nozzle in the gantry. By means of magnets positioned adjacent to the nozzle, the beam path, Xp, of a hadron beam 1h may be deviated with respect to the path axis, Xn, within a cone centred on the path axis and having the nozzle as apex (as depicted, for example, in FIG. 4A). Advantageously, this may allow a volume of target tissue centred on the isocentre to be treated by a hadron beam without changing the position of the nozzle within the gantry. The same applies to fixed nozzles with the difference that the angular position of the path axis may be fixed.

A target tissue to be treated by a hadron beam in a device provided with a gantry must generally be positioned near the isocentre. Accordingly, the couch or any other support for the patient may be moved; for example, it may typically be translated over a horizontal plane (X, Z), wherein X is a horizontal axis normal to the horizontal axis, Z, and translated over a vertical axis, Y, normal to X and Z, and may also be rotated about any of the axes X, Y, Z, so that a central area of the target tissue may be positioned at the isocentre.

To assist in the correct positioning of a patient with respect to the nozzle 12n according to a treatment plan previously established, the beam delivery system may comprise imaging means. For example, a conventional X-ray radiography system may be used to image an imaging volume, Vp, comprising the target tissue 40. The obtained images may be compared with corresponding images collected previously during the establishment of the treatment plan.

Depending on the pre-established treatment plan, a hadron treatment may comprise delivery of a hadron beam to a target tissue in various forms, including, for example, pencil beam, single scattering, double scattering, uniform scattering, and the like. Embodiments of the present disclosure may apply to all hadron therapy techniques. FIG. 4B illustrates schematically a pencil beam technique of delivery. As depicted in FIG. 4B, hadron beam of initial energy, Ek,1, may be directed to a first target spot 40s1,1, during a pre-established delivery time. The hadron beam may then be moved to a second target spot 40s1,2, during a pre-established delivery time. The process may be repeated on a sequence of target spots 40s1,j to scan a first iso-energy treatment volume, Vt1, following a pre-established scanning path. A second iso-energy treatment volume, Vt2, may be scanned spot-by-spot following a similar scanning path with a hadron beam of initial energy, Ek,2. As many iso-energy treatment volumes, Vti, as necessary to treat a given target tissue 40 may thus be irradiated following a similar scanning path. A scanning path may include several passages over a same scanning spot 40si,j. The iso-energy treatment volumes, Vti, may be volumes of target tissues which may be treated with a hadron beam of initial energy, Ek,i. The iso-energy treatment volumes, Vti, may be slice shaped, with a thickness corresponding approximately to the breadths of the Bragg peaks at the values of the initial energy, Ek,i, of the corresponding hadron beams, and with main surfaces of area only limited by the opening angle of the cone centred on the path axis, Xn, enclosing the beam paths, Xp, available for a given position of the nozzle in the gantry or in a fixed nozzle device. In embodiments with a homogeneous target tissue, the main surfaces may be substantially planar as illustrated in FIG. 4B. In embodiments where both target tissue 40 and upstream tissues 41-43 are not homogeneous in nature and thickness, the main surfaces of an iso-energy volume, Vti, may be bumpy. The egg-shaped volumes in FIG. 4B schematically illustrate the volumes of target tissue receiving a therapeutic dose of hadron by exposure of one target spot 40si,j to a beam of initial energy Ek,i.

The dose, D, delivered to a target tissue 40 is illustrated in FIG. 4C. As discussed supra, the dose delivered during a treatment session is usually of the order of one to several Grays (Gy). It may depend on the doses delivered to each target spot 40si,j, of each iso-energy treatment volume, Vti. The dose delivered to each target spot 40si,j may depend on the intensity, I, of the hadron beam and on the irradiation time tij on said target spot. The dose, Dij, delivered to a target spot 40si,j may therefore be the integral, Dij=∫I dt, over the irradiation time tij. A typical dose, Dij, delivered to a target spot 40si,j is generally of the order of 0.1-20 cGy. The dose, Di, delivered to an iso-energy treatment volume, Vti, may be the sum over the n target spots scanned in said iso-energy treatment volume of the doses, Dij, delivered to each target spot, Di=Σ Dij, for j=1 to n. The total dose, D, delivered to a target tissue 40 may thus be the sum over the p irradiated iso-energy treatment volumes, Vti, of the doses, Di, delivered to each energy treatment volume, D=Σ Di, for i=1 to p. The dose, D, of hadrons delivered to a target tissue may therefore be controlled over a broad range of values by controlling one or more of the intensity, I, of the hadron beam, the total irradiation time tij of each target spot 40si,j, and/or the number of irradiated target spots 40si,j. Once a patient is positioned such that the target tissue 40 to be treated is located at the approximate position of the isocentre, the duration of a hadron treatment session may depend on the values of:
the irradiation time, tij, of each target spot 40si,j,
the scanning time, Δti, for directing the hadron beam from a target spot 40si,j to an adjacent target spot 40si(j+1) of a same iso-energy treatment volume, Vti,
the number n of target spots 40si,j scanned in each iso-energy treatment volume, Vti,
the time, ΔtVi, required for passing from a last target spot 40si,n scanned in an iso-energy treatment volume, Vti, to a first target spot 40s(i+1),1 of the next iso-energy treatment volume, Vt(i+1), and/or
the number of iso-energy treatment volumes, Vti, in which a target tissue 40 may be enclosed.

The irradiation time, tij, of a target spot 40si,j is generally of the order of 1-20 ms. The scanning time, Δti, between successive target spots in a same iso-energy treatment volume may be very short, of the order of 1 ms. The time, ΔtVi, required for passing from one iso-energy treatment volume, Vti, to a subsequent iso-energy treatment volume, Vt(i+1), may be slightly longer because, for example, it may require changing the initial energy, Ek, of the hadron beam. The time required for passing from one volume to a subsequent volume is generally of the order of 1-2 s.

As evidenced in FIGS. 2A and 2B, an accurate determination of the initial energy, Ek, of a hadron beam may be important because, if the position of the Bragg peak does not correspond to the actual position of the target tissue 40, substantial doses of hadrons may be delivered to healthy, sometimes vital, organs and may possibly endanger the health of a patient. The position of the Bragg peak may depend on the initial energy, Ek, of the hadron beam and/or on the nature and thicknesses of the traversed tissues. Besides determining the position of the target tissue within a patient, the computation of the initial energy, Ek, of a hadron beam yielding a position of the Bragg peak corresponding to the precise position of the target tissue may also require the preliminary characterization of the tissues traversed until reaching the target tissue 40. This characterization may be performed during a treatment plan established before (e.g., generally several days before) the actual hadron treatment. The actual hadron treatment may be divided in several sessions distributed over several weeks. A typical treatment plan may start by the acquisition of data, e.g., generally in the form of images of the subject of interest with a CT scan. The images thus acquired by a CT scan may be characterized, for example, by:
identifying the nature of the tissues represented on the images as a function of the X-rays absorption power of the tissues, e.g., based on the comparison of shades of grey of each tissue with a known grey scale; for example, a tissue may be one of fat, bone, muscle, water, air, or the like;
measuring the positions and thicknesses of each tissue along one or more hadron beam paths, Xp, from the skin to the target tissue;
based on their respective nature, attributing to each identified tissue a corresponding hadron stopping power ratio (HSPR);
calculating a tissue water equivalent path length, WEPLm, of each tissue m, with m=40 to 44 in the illustrated examples of FIGS. 2A and 2B, upstream of and including the target tissue, based on their respective HSPR and thicknesses;
adding the determined WEPLm of all tissues m to yield a WEPL40s of a target spot 40s located in the target tissue 40, said WEPL40s corresponding to the distance travelled by hadron beam from the skin to the target spot 40s; and
based on the WEPL40s, calculating the initial energy Ek of a hadron beam required for positioning the Bragg peak of the hadron beam at the target spot 40s.
   Said process steps may be repeated for several target spots defining the target tissue.

### Magnetic Resonance Imaging Device

A magnetic resonance imaging device 2 (MRI) generally implements a medical imaging technique based on the interactions of excitable atoms present in an organic tissue of a subject of interest with electromagnetic fields. When placed in a strong main magnetic field, B0, the spins of the nuclei of said excitable atoms typically precess around an axis aligned with the main magnetic field, B0, resulting in a net polarization at rest that is parallel to the main magnetic field, B0. The application of a pulse of radio frequency (RF) exciting magnetic field, B1, at the frequency of resonance, fL, called the Larmor frequency, of the excitable atoms in said main magnetic field, B0, may excite said atoms by tipping the net polarization vector sideways (e.g., with a so-called 90° pulse, B1-90) or to angles greater than 90° and even reverse it at 180° (e.g., with a so-called 180° pulse, B1-180). When the RF electromagnetic pulse is turned off, the spins of the nuclei of the excitable atoms generally return progressively to an equilibrium state yielding the net polarization at rest. During relaxation, the transverse vector component of the spins typically produces an oscillating magnetic field inducing a signal, which may be collected by antennas 2a located in close proximity to the anatomy under examination.

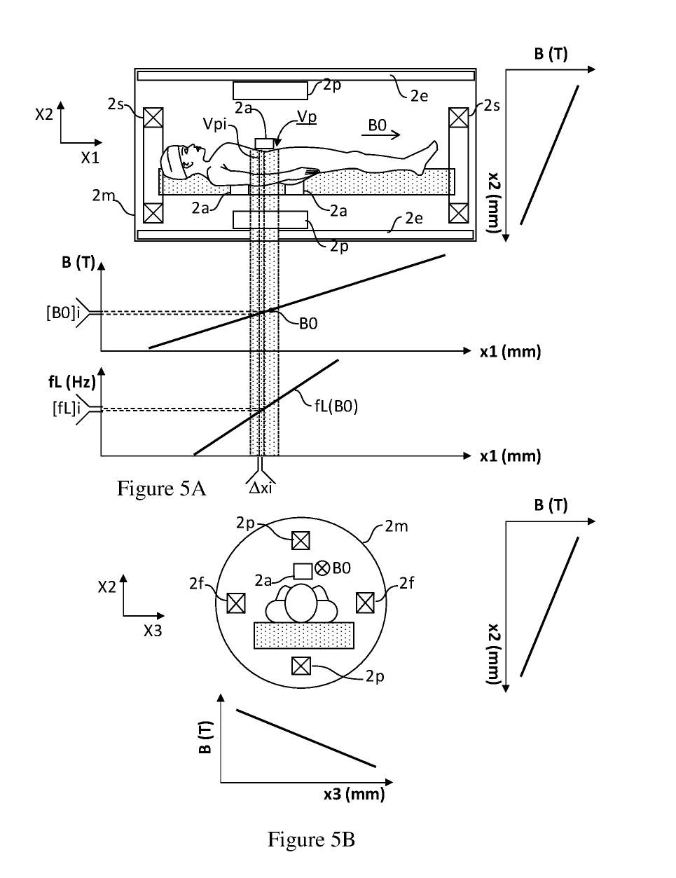

As shown in FIGS. 5A, 5B, 6A, and 6B, an MRI 2 usually comprises a main magnet unit 2m for creating a uniform main magnetic field, B0; radiofrequency (RF) excitation coils 2e for creating the RF-exciting magnetic field, B1; X1-, X2-, and X3-gradient coils, 2s, 2p, 2f, for creating magnetic gradients along the first, second, and third directions X1, X2, and X3, respectively; and antennas 2a, for receiving RF-signals emitted by excited atoms as they relax from their excited state back to their rest state. The main magnet may produce the main magnetic field, B0, and may be a permanent magnet or an electro-magnet (e.g., a supra-conductive magnet or not). An example of a suitable MRI includes, but is not limited to, a device described in EP Pat. No. 0186238, the entire disclosure of which is incorporated herein by reference as representative of an MRI used in the present disclosure.

As illustrated in FIG. 5A, an imaging slice or layer, Vpi, of thickness, Δxi, normal to the first direction, X1, can be selected by creating a magnetic field gradient along the first direction, X1. In FIG. 5A, the first direction, X1, is parallel to the axis Z defined by the lying position of the patient, yielding slices normal to said axis Z. In some embodiments, the first direction, X1, may be any direction, e.g., transverse to the axis Z, with slices extending at an angle with respect to the patient. As further shown in FIG. 5A, because the Larmor frequency, fL, of an excitable atom generally depends on the magnitude of the magnetic field it is exposed to, sending pulses of RF exciting magnetic field, B1, at a frequency range, [fL]i, may excite exclusively the excitable atoms which are exposed to a magnetic field range, [B0]i, which may be located in a slice or layer, Vpi, of thickness, Δxi. By varying the frequency bandwidth, [fL]i, of the pulses of RF exciting magnetic field, B1, the width, Δxi, and position of an imaging layer, Vpi, may be controlled. By repeating this operation on successive imaging layers, Vpi, an imaging volume, Vp, may be characterized and imaged.

To localize the spatial origin of the signals received by the antennas on a plane normal to the first direction, X1, magnetic gradients may be created successively along second and third directions, X2, X3, wherein X1 ⊥ X2 ⊥ X3, by activating the X2-, and X3-gradient coils 2p, 2f, as illustrated in FIG. 5B. Said gradients may provoke a phase gradient, Δφ, and a frequency gradient, Δf, in the spins of the excited nuclei as they relax, which may allow spatial encoding of the received signals in the second and third directions, X2, X3. A two-dimensional matrix may thus be acquired, producing k-space data, and an MR image may be created by performing a two-dimensional inverse Fourier transform. Other modes of acquiring and creating an MR image may be utilized concurrently with or alternatively to the mode described above.

The main magnetic field, B0, may be between 0.2 T and 7 T, e.g., between 1 T and 4 T. The radiofrequency (RF) excitation coils 2e may generate a magnetic field at a frequency range, [fL]i, around the Larmor frequencies, fL, of the atoms comprised within a slice of thickness, Δxi, and exposed to a main magnetic field range [B0i]. For atoms of hydrogen, the Larmor frequency per magnetic strength unit is approximately fL/B=42.6 MHz T⁻¹. For example, for hydrogen atoms exposed to a main magnetic field, B0=2 T, the Larmor frequency is approximately fL=85.2 MHz.

The MRI may be any of a closed-bore, open-bore, or wide-bore MRI type. A typical closed-bore MRI has a magnetic strength of 1.0 T through 3.0 T with a bore diameter of the order of 60 cm. An open-bore MRI, as illustrated in FIGS. 6A and 6B, has typically two main magnet poles 2m separated by a gap for accommodating a patient in a lying position, sitting position, or any other position suitable for imaging an imaging volume, Vp. The magnetic field of an open-bore MRI is usually between 0.2 T and 1.0 T. A wide-bore MRI is a kind of closed-bore MRI having a larger diameter.

### Hadron Therapy Device+MRI

As discussed previously with reference to FIG. 2B, the position and morphology of a target tissue 40 may evolve between a time, t0, of establishment of a treatment plan and a time, t1=t0+Δt3, of a treatment session, which may be separated by several days or weeks. A target spot 40si,j identified in the treatment plan as belonging to the target tissue 40p may not belong to the target tissue 40 anymore at the time, t0+Δt3, of the treatment session. The irradiation of said target spot may harm healthy tissues 43 instead of target tissues 40.

To avoid such incidents, a hadron therapy device (PT) 1 may be coupled to an imaging device, such as a magnetic resonance imaging device (MRI) 2. Such coupling may raise a number of challenges to overcome. For example, the correction of a hadron beam path, Xp, within a strong magnetic field, B0, of the MRI is a well-researched problem with proposed solutions.

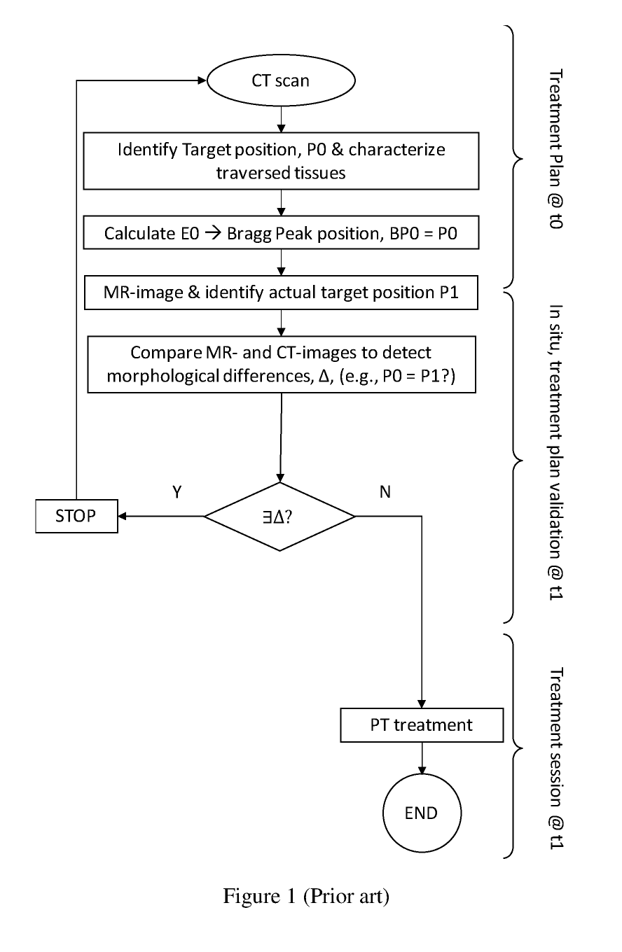

A PT-MRI apparatus may allow the morphologies and positions of the target tissue and surrounding tissues to be visualized, for example, on the day, t0+Δt3, of the treatment session for comparison with the corresponding morphologies and positions acquired during the establishment of a treatment plan at time, t0. As illustrated in the flowchart of FIG. 1, in cases having a discrepancy of the tissues morphologies and positions between the establishment of the treatment plan at time, t0, and the treatment session at time, t0+Δt3, the treatment session may be interrupted and a new treatment plan may be established with the definition of new target spots corresponding to the actual target tissue 40 to be irradiated by hadron beams of corrected energies and directions (in the example of FIG. 1, this procedure is represented by diamond box “∃Δ?”→Y→“STOP”). This represents a major improvement over carrying out a hadron therapy session based solely on information collected during the establishment of the treatment plan at time, t0, which may be obsolete at the time, t0+Δt3, of the treatment session.

Embodiments of the present disclosure may further improve the efficacy of a PT-MRI apparatus by providing the information required for correcting in situ the initial energies, Ek, and beam path, Xp, directions of the hadron beams, in case a change of morphology or position of the target tissue were detected. This may allow the treatment session to take place in spite of any changes detected in the target tissue 40.

The MRI used in embodiments of the present disclosure may be any of a closed-bore, open-bore, or wide-bore MRI type described above. An open MRI may provide open space in the gap separating the two main magnet poles 2m for orienting a hadron beam in almost any direction. Alternatively, openings or windows 2w transparent to hadrons may be provided on the main magnet units, as illustrated in the example of FIG. 6A. This configuration may allow the hadron beam to be parallel to B0. In another embodiment, a hadron beam may be oriented through the cavity of the tunnel formed by a closed bore MRI, or an annular window transparent to hadrons may extend parallel to a gantry substantially normal to the axis Z, over a wall of said tunnel, such that hadron beams may reach a target tissue with different angles. In embodiments where a fixed nozzle is used, the size of such opening or window may be reduced accordingly.

### Prompt-γ System

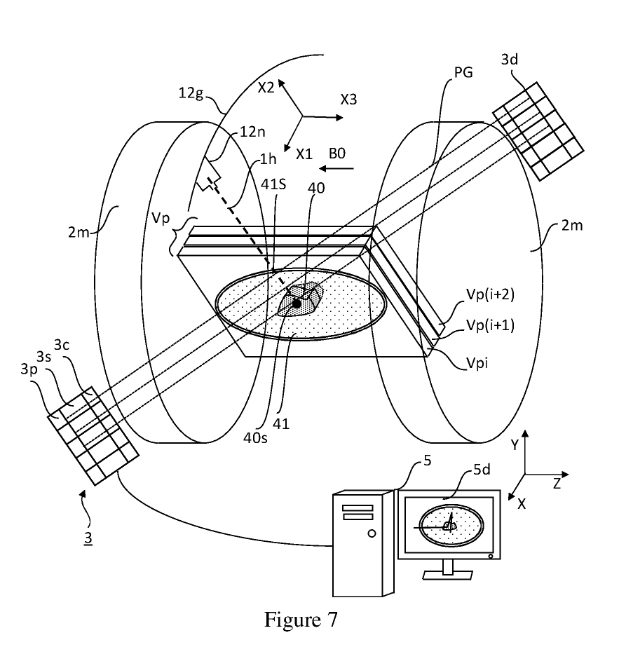

FIG. 7 illustrates an example of a medical apparatus comprising a hadron therapy device 1, a magnetic resonance imaging device (MRI) 2, and a prompt-γ (PG) system 3.

The PG system may comprise a detector 3d configured for detecting a signal generated by a hadron beam 1h upon interaction with the subject of interest. The hadron beam may be a treatment hadron beam having an initial beam energy E0, for example, between 0 and 230 MeV. The hadron beam may be directed along a beam path towards a one or more target spots 40si,j located inside the target tissue 40. For example, the beam path of the hadron beam may travel from the nozzle 12n of the beam delivery system and through a subject of interest to a target spot 40s. The beam path may also cross an outer surface 41S of the subject of interest and one or more tissues m with m=40-44 as shown in the example of FIGS. 2A, 2B, and 2C.

A hadron beam that crosses material (e.g., tissues) generally loses a part of its energy all along its beam path. The loss is typically due to the interactions of the hadrons with the electrons of the tissues traversed and to the interactions with the atomic nuclei of the tissues traversed. The loss may be proportional to the thickness Lm of the tissue m traversed and may depend on the nature of the tissue m. In hadron therapy, the tissue traversed by a hadron beam may be, for example, skin, fat, muscle, bone, air, water (and/or blood), organ, tumour, or the like. A part of the energy lost along the beam path by the hadron beam is generally due to inelastic interactions (i.e., collisions) between the hadrons of the hadron beam and atomic nuclei of the tissues traversed. The interactions may excite the atomic nuclei, bringing them in a higher energy state than the ground state before interactions. The atomic nuclei typically rapidly return to their ground state by emitting a prompt γ-ray (PG). The emission of PG typically occurs along the beam path, and its intensity may depend on the probability of interaction of a hadron with an atomic nuclei and, therefore, on the energy of the hadron. The PG emission profile usually follows a curve correlated with the Bragg curve. Note that the position of the fall-off of the PG may not be exactly the same as the position of the dose fall-off of the Bragg curve. For example, the PG peak may occur a few (e.g., 2-3) mm before the Bragg peak. The emission spectrum of PG is generally dominated by several discrete lines from specific nuclear de-excitation, e.g., in the range 1-15 MeV, and may be isotropic. Because of their high characteristic energies, PG may escape the subject of interest with high probability, and they may be detected with a PG system, allowing the possibility to retrieve the beam penetration depth (i.e., position of the Bragg peak) within the subject of interest.

The detector 3d of the PG system may detect a signal generated by the PG emitted along the beam path. This signal may allow computing the position of the Bragg peak of the hadron beam. Several techniques, depending on the signal acquired, may be used to measure the position of the Bragg peak:
(A) PG imaging technique, which uses the incidence of the detected γ to determine its emission point;
(B) PG timing technique, which uses the time-of-flight of the detected γ to determine the distance from the detector to the position where the γ ray has been emitted along the hadron beam; and/or
(C) PG spectroscopy technique, which uses the distribution of energy of the detected γ emitted at a given position along the beam path to retrieve the energy of the hadron beam at the given position.

The computation of the position of the Bragg peak within the subject of interest may be performed by simulating the PG emission of a simulated hadron beam. The simulation may then be compared with the measured emission and, in case of discrepancy, be corrected.

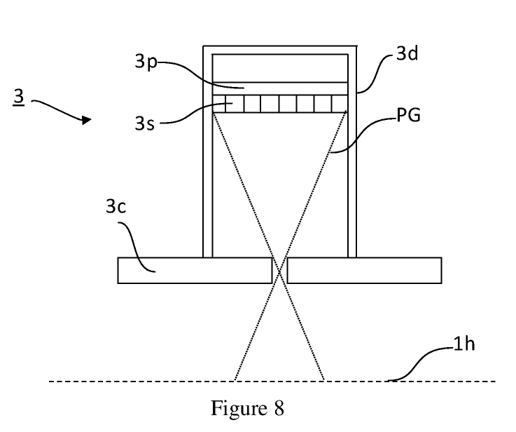

FIG. 8 shows an example of a detector 3d of the PG system 3 comprising a collimator 3c, a scintillator 3s, and a photon counting device 3p. The scintillator may comprise a scintillating material which interacts with PG to generate visible photons. The scintillator may be segmented or not. In embodiments where the scintillator is segmented, each segment may correspond to a portion of the field of view of the detector. The collimator may comprise a longitudinal slit-shaped opening 3o. For example, the opening 3o of the collimator 3c may be configured to select the PG emitted normally to the beam path. The photon counting device 3p may also comprise a photomultiplier. A PG, selected by the collimator, may interact with the scintillator. Then, visible photons may be multiplied with the photomultiplier to increase the signal that is acquired with the photon counting device. An example of a suitable PG detector includes, but is not limited to, a device described in European Pat. application No. 2977083A1, the entire disclosure of which is incorporated herein by reference as representative of a PG detector used in the present disclosure.

The medical apparatus according to one embodiments of the present disclosure may comprise:
(A) a hadron therapy device comprising a hadron source adapted for directing a hadron beam having an initial beam energy, E0, along a beam path to a target spot 40s located inside a subject of interest;
(B) a MRI for acquiring a magnetic resonance (MR) image within an imaging volume, Vp, comprising the target spot;
(C) a PG system adapted for acquiring a signal generated by the hadron beam; and
(D) a controller configured for:
   computing an actual position, BP1, of the Bragg peak of said hadron beam, based on the signal acquired by the PG system; and
   locating the actual position, BP1, of the Bragg peak on the MR image of the imaging volume, Vp, acquired with the MRI along the beam path from an outer surface 41S of the subject of interest to the target spot 40s.

The water equivalent path length, and thus the energy, of a treatment hadron beam to a target tissue may change between the establishment of a treatment plan and a treatment session. For example, a patient having a cerebral tumour may have a flu that fills the sinus with water instead of air. The presence of the water may modify the water equivalent path length computed during the treatment plan. Accordingly, the target spots located behind the sinus and along the beam path may not be correctly irradiated. An apparatus of the present disclosure may allow for correcting the energy of target spots during the treatment session, thus minimizing or preventing the irradiation of healthy tissues instead of target tissue. The apparatus may measure the position of the Bragg peak of one or some target spots (e.g., 1-20) of a same iso-energy volume (e.g., typically comprising 100 target spots) and locate the measures on an MR image. From that, a controller may calculate if modifications have been occurred between the treatment plan and the treatment session. It may then adapt the treatment session for the remaining target spots.

In some embodiments, the MR image provided by the MRI and the signal generated by the hadron beam provided by the PG system may be acquired simultaneously or with a short delay. The two measures may thus be representative of the same configuration of the tissues.

The plan of the MR image may comprise the beam path of the (imaging) hadron beam. The MR image may be used to (at least in part) determine the nature of the tissues m traversed by the hadron beam and to determine the thicknesses Lm of the tissues m traversed by the hadron beam. In some embodiments, then, the MRI may image the plan in which the imaging hadron beam passes.

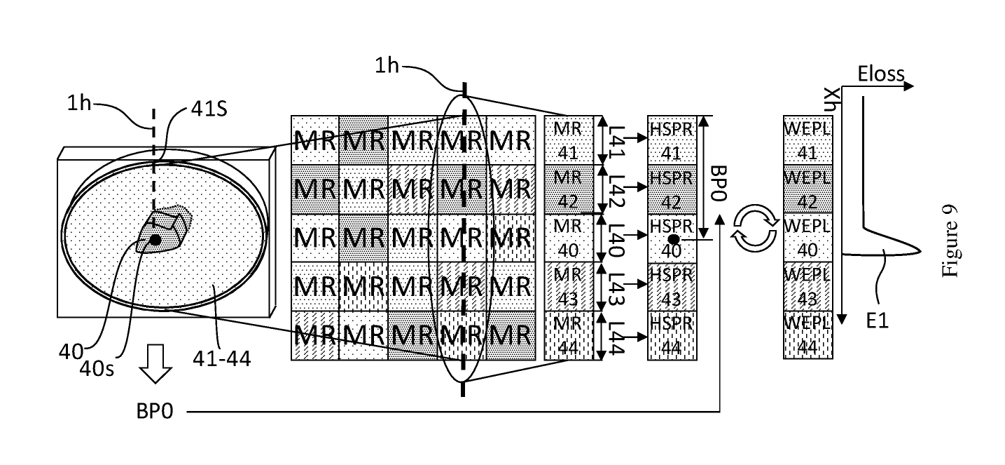

As illustrated in FIG. 9, the controller 5 may acquire the signal provided by the PG system and the MR image provided by the MRI. The MR image may be used to identify the position of the outer surface 41S of the subject of interest. The tissue traversed by the hadron beam may be selected on the MR image. The controller may determine or estimate:
the nature of the tissues m traversed by the hadron beam;
an HSPR,m of each tissue m; and/or
the thickness, Lm of the tissues m.

The controller may then use the signal provided by the PG system and the information from the MR image to compute the actual position, BP1 of the Bragg Peak of the hadron beam. In some embodiments, the computation may be an iterative process. For example, the emission of PG of a hadron beam in the traversed tissue may be simulated. The simulation may be compared to the measured signal. In case of a difference, the simulation may be adapted (for example, by modifying the estimated HSPR,m of some tissues m, modifying the estimated thickness, Lm, or the like), and a hadron beam may again be simulated. This procedure may be performed until the simulation and the measured signal are (or at least are nearly) the same. In some embodiments, the computation may be performed until the difference between the simulation and the measured signal is smaller than a given tolerance.

Alternatively or concurrently, the initial HSPR,m, Lm, and nature of the tissues m used in the simulation may be those computed during the treatment plan, which are usually more accurate.

The controller may then compare the actual position, BP1, of the Bragg Peak with the actual position, P1, of the target spot 40s targeted by the hadron beam.

The position of the Bragg peak generally depends on the initial energy E0 of a hadron beam and on a water equivalent path length of the hadron beam. Knowing the position of the Bragg peak and the initial energy E0 of the hadron beam may allow for computing the water equivalent path length WEPL40s corresponding to the water equivalent path length between the outer surface 41S of the subject of interest and the target spot 40s. The energy lost in the air before the outer surface of the subject of interest is often negligible.

In case the actual position, BP1, and the actual position, P1, of the target spot 40s are offset by a distance greater than a given tolerance, the controller may compute the water equivalent path lengths (WEPLm) of each tissue m, crossed by the beam path and between the outer surface 41S and the target spot 40s.

For example, the computation may use data extracted from MR images (the nature of the tissues m, HSPR,m, thickness, Lm) and the WEPL40s obtained from PG system. The controller may also use data computed during the treatment plan to improve the accuracy and the speed of the computation. For example, the controller may identify the (e.g., morphological) difference between the MR image of the treatment session and the CT (and/or MR) image of the treatment plan, thus indicating the parameters that have to be changed in the computation.

The tolerance may be less than ±10 mm, e.g., ±5 mm or ±3 mm. In practice, a person of ordinary skill in the art may estimate range uncertainties in hadron therapy by applying, e.g., Monte Carlo, simulations. The tolerance may also be dependent on the expected precision of the detector for the target spot measured, which may depend on: the number of hadrons stopping on the target spot, the distance, the beam energy, the nature of the tissue, and the like. The tolerance thus may be dependent on the subject of interest and/or on the target spot.

An apparatus according to some embodiments of the present disclosure may further comprise a display 5d, and the controller may be configured to represent, on a same coordinate scale, the MR image obtained from the MRI and the position of the Bragg peak obtained from the PG system.

The WEPLm may be used to correct the planned initial beam energy E0 of target spot 40s. The energy E0 may be increased or decreased to a corrected initial energy E1 such that the position of the Bragg peak of the hadron beam corresponds to the position of the target spot 40s. The corrected initial beam energy E1≡E1,i may then be suitable for matching the positions of the Bragg peak of said hadron beam with the positions of all the other target spots 40si,j located in a same iso-energy volume, Vti.

In some embodiments, the WEPLm may be computed for several spots of the same iso-energy volume, Vti in order to increase the reliability of the computation and to avoid local effects (such as the above example of the water in the sinus).

The computation of the corrected initial beam energy E1 may thus be performed on the basis of some target spots within a same iso-energy volume, Vti. As discussed with respect to the example of FIG. 4C, this may be achieved either by irradiating few target spots, e.g., irradiating between 1% and 40% of the target spots of an iso-energy layer, Vti, e.g., between 5% and 30% or between 10% and 20%. The dose delivered by the hadron beam directed towards these spots may thus be insufficient to treat the target tissue because, in case of a change of morphology of the tissues, a full therapeutic dose reaching healthy tissues may be extremely detrimental to the health of a patient. In these conditions, then, the validation of the treatment plan according to embodiments of the present disclosure is generally safe for the patient, even if a correction of the initial energy is required. In some embodiments, the corrected initial energy, E1, may be used during the treatment session to treat all the target spots 40si,j of an iso-energy volume, Vti. The initial energies required for treating target spots, 40(i+1),j, etc., in subsequent iso-energy volumes, Vt(i+1), etc., may either be extrapolated from the initial energy, E1, and/or determined for the iso-energy volume, Vti, or, alternatively or additionally, a selection of target spots 40(i+1),j, etc., of the subsequent energy volumes, Vt(i+1), etc., may be tested as described above.

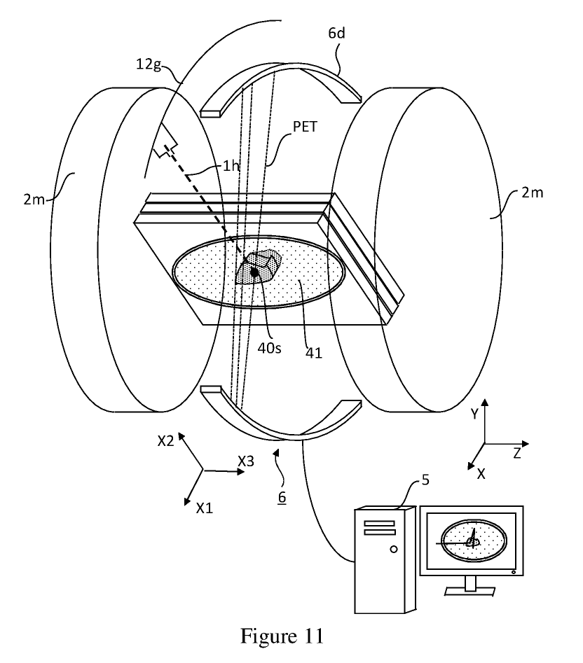

Alternatively, as illustrated in FIG. 11, the PG system may be replaced by or paired with a positron emission tomography (PET) scan 6. A PET scan is a device for imaging in 3D the concentration of β⁺ (positron) emitter located along the hadron beam path in the subject of interest. A small fraction of the hadrons of the hadron beam may create positron emitting isotopes (for example, ¹¹C, ¹³N, ¹⁵O) through interactions with the atomic nuclei of the tissues traversed. These radio-active isotopes generally decay with emission of a positron which may annihilate with an electron, leading to the emission of two gamma photons emitted in coincidence. The detector 6d of the PET scan may detect the source of emission of these two gamma photons and therefore measure the concentration of β⁺ emitter. The concentration of β⁺ emitter may be related to the beam path of the hadron beam.

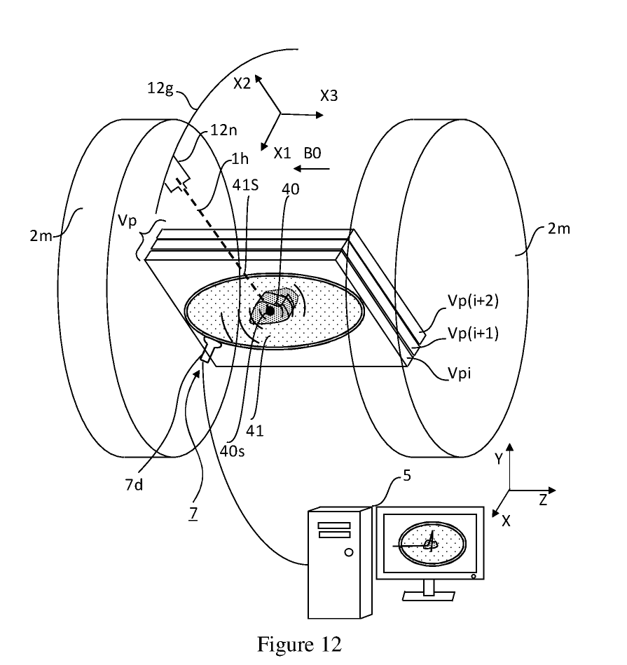

In yet another alternative, an ultrasound system may replace or be paired with the PG system. FIG. 12 shows an example of ultrasonic system 7 comprising an ultrasonic detector 7d. An example of a suitable ultrasonic system includes, but is not limited to, a device described in Assmann, W., Kellnberger, S., Reinhardt, S., Lehrack, S., Edlich, A., Thirolf, P. G., Parodi, K. (2015). Ionoacoustic characterization of the proton Bragg peak with submillimeter accuracy. Medical Physics, 42(2), 567-74. http://doi.org/10.1118/1.4905047, the entire disclosure of which is incorporated herein by reference as representative of an ultrasound system used in the present disclosure.

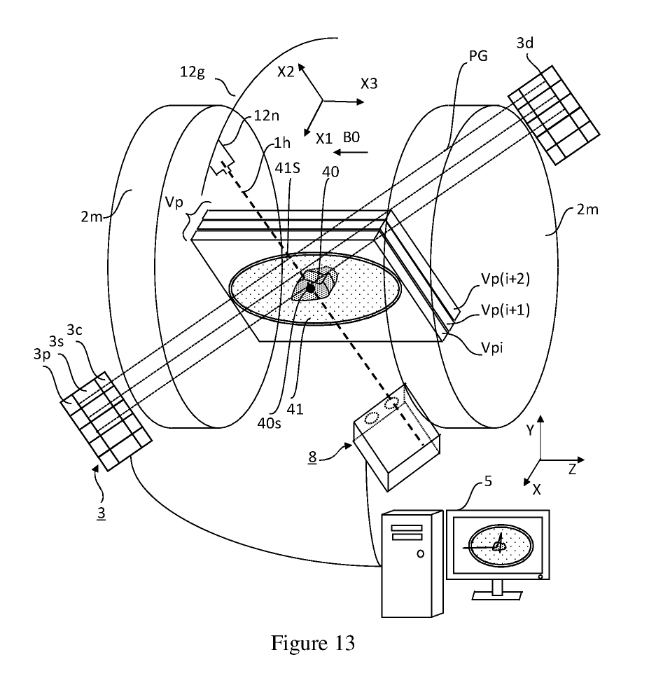

A medical apparatus according to embodiments of the present disclosure may also comprise a hadron radiography system (HRS), e.g., a proton radiography system (PRS) 8, as shown in FIG. 13. An HRS uses an imaging hadron beam that crosses the subject of interest (and the target spot) and may measure the water equivalent path length, WEPL,HRS, of the subject of interest crossed by the hadron beam. This WEPL,HRS may provide additional information on the position of the Bragg peak and may be used to improve the WEPLm determination. Accordingly, it may allow for an improvement of the range determination of the hadron beam. The detector of the HRS may be one of the following detectors: a range telescope, a calorimeter, a spectrometer, or the like.

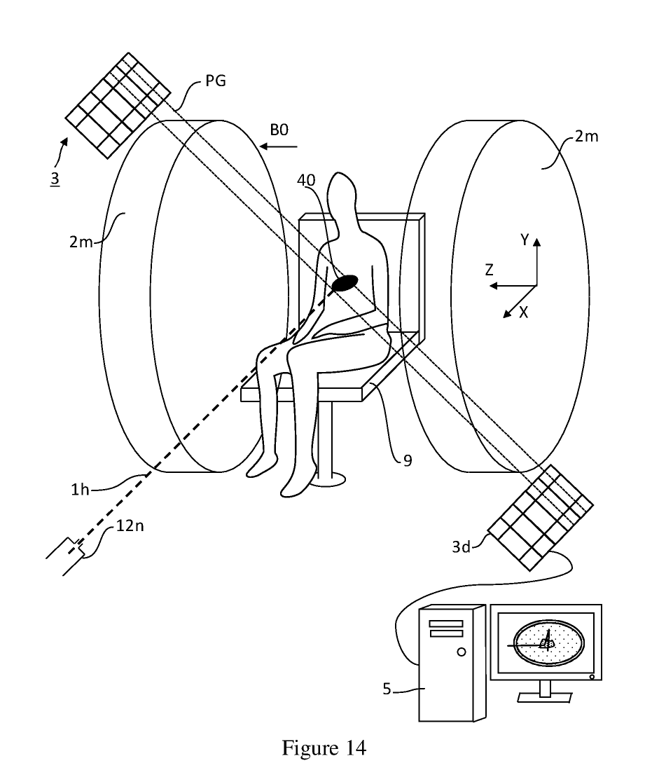

FIG. 14 illustrates an example hadron therapy device according to one embodiment of the present disclosure further comprising a support 9 for supporting a patient in a non-supine position. A low uncertainty on the position of the target tissue 40s may permit large morphological differences between the establishment of the treatment plan and the treatment session. In this context, treating a patient in a non-supine position may be advantageous because it generally does not require a gantry. The beam nozzle may thus be fixed, and the cost of the apparatus may be reduced. The apparatus according to the embodiment depicted in FIG. 14 may also comprise an HRS.

According to a second aspect, the present disclosure relates to a method for locating the Bragg peak of a hadron beam having an initial beam energy, E0 and being emitted along a beam path to a target spot 40s within a target tissue 40. The location of the Bragg peak of a hadron beam with respect to the target spot may allow for verifying a treatment plan previously established.

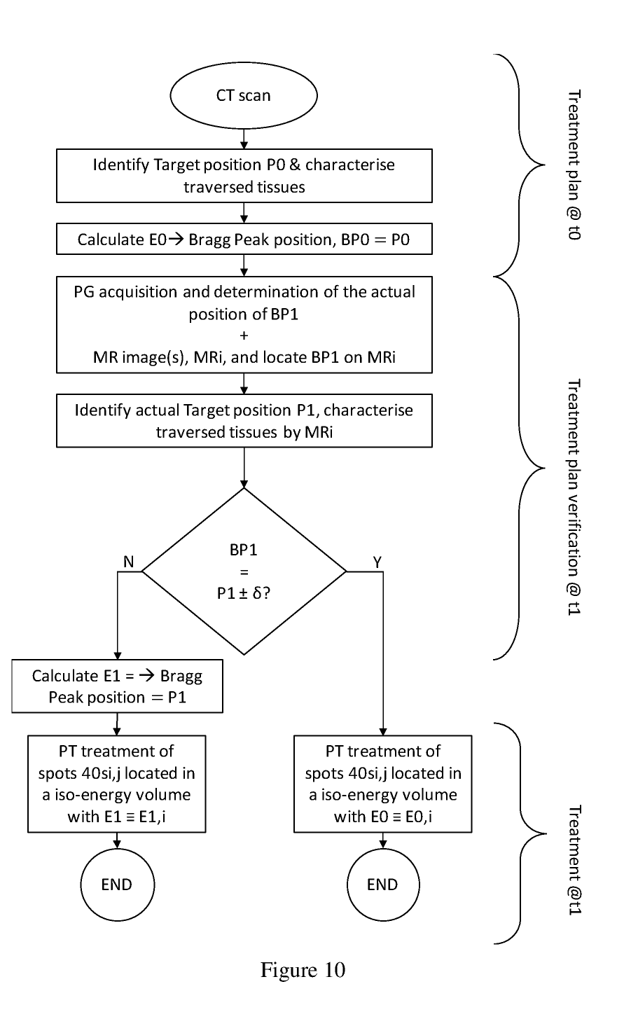

FIG. 10 illustrates an example flowchart of such a method according to an embodiment of the present disclosure. First, a classical treatment plan may be established at a time t0, using a CT scan (and/or an MR image) described above. A typical treatment plan may provide images of the subject of interest with a CT scan. The images may permit identifying the position, P0, of a target spot of the target tissue 40 and characterizing the tissues traversed by the hadron beam. A treatment plan system may then compute the initial beam energy, E0, such that the position, BP0, of the Bragg peak corresponds to the position, P0, of the target sport of the target tissue. These operations may be repeated for several target spots 40si,j.

This method according to the present disclosure may be performed, for example, during a treatment session. As illustrated in FIG. 10, the localisation of the Bragg peak of a hadron beam is the first step of the method. A magnetic resonance (MR) imaging of an imaging volume, Vp, comprising a target spot 40s may be performed and an MR image acquired. Then, a PG system may detect and acquire a signal generated by a hadron beam having an initial beam energy, E0 and being emitted, along the beam path to the target spot 40s.

As described above, the signal acquired by the PG system may allow for computing the actual position, BP1, of the Bragg peak of the hadron beam. The actual position BP1 may then be located on the MR image.

This method of the present disclosure may also comprise a comparison of the actual position BP1 of the Bragg peak with the actual position, P1, of the target spot 40s determined from the MR image during the verification of the treatment plan, for example, during a treatment session at the time t1 later than t0.

When the actual position, BP1, of the Bragg peak, and the actual position, P1, of the target spot 40s are offset by a distance greater than a given tolerance δ, a correction of the initial energy of the hadron beam may be performed. To achieve this correction, the water equivalent path lengths WEPLm of each tissue m crossed by the beam path and between the outer surface 41S and the target spot 40s may be computed. For example, the computation may be based on the thickness Lm and nature of each tissue m determined on the MR image. The computation may also use the water equivalent path length WEPL40s corresponding to the distance between the outer surface 41S and the target spot 40s. The WEPL40s may be determined by the PG system using the actual position BP0 of the Bragg peak.

The tolerance 6 on the offset between the actual position, BP1, of the Bragg peak, and the actual position, P1, of the target spot 40s may be less than ±10 mm, e.g., ±5 mm or ±3 mm.

The planned initial beam energy E0 of target spot 40s may then be corrected to a corrected initial beam energy E1, suitable for matching the positions of the Bragg peak of said hadron beam with the actual position of target spot 40s. This energy may also be suitable for all target spots 40si,j located in a same iso-energy volume, Vti, then the target spot 40s.

In some embodiments, the magnetic resonance image obtained from the MRI and the actual position, BP1, of the Bragg peak obtained from the PG system may be represented on a display 5d on a same coordinate scale.

As described above, methods and apparatuses according to some embodiments of the present disclosure may be used to compute the position, BP1, of the Bragg peak of the hadron beam from the signal generated by the emission of PG of one or several target spots 40si,j and acquired by the PG system. The computation may be performed during a treatment session. The total treatment usually comprises several treatment sessions, and the time between the first and the treatment session may be separated by, for example, several days or weeks. In some embodiments, the computation may thus be performed during several treatment sessions at time t0+Δt1, t0+Δt2, t1=t0+Δt3 for at least a portion of the target spots 40si,j.

The measurement(s) acquired during the treatment sessions at time t0+Δt1, t0+Δt2, t1=t0+Δt3 may allow computing an evolution of the actual position, BP1, of the Bragg peak of one or more target spots 40si,j. The evolution may permit observation of general trends of modifications of the morphology and/or position of the target tissue (or surrounding tissue). When such trends are observed and exceed predetermined limits, a new treatment plan may be established. The trends may also be used to extrapolate a treatment that will be delivered later.

In some embodiments, the magnetic resonance (MR) imaging and the emission of a hadron beam are done in the same room.

In some embodiments, a method according to the present disclosure may be performed with a medical apparatus according to the present disclosure.

Embodiments of the present disclosure may thus reduce the range uncertainty of a hadron beam. The use of the PG system may allow for measuring the position of the Bragg peak of a hadron beam within a subject of interest. The MRI may then provides images that help identify the nature and thickness of the tissues traversed by the hadron beam and identify the outer surface of the subject of interest. Accordingly, the signal from the PG system and the MR image may be represented on the same scale. This information may be used to check a treatment plan during a treatment session, thus reducing the risk of incorrect treatment and improving the quality (e.g., adaptation of the energy) and precision (e.g., lower uncertainty) of the treatment.

1.-15. (canceled)

16. A medical apparatus, comprising:
a hadron therapy device including a hadron source adapted to direct a hadron beam with an initial beam energy along a beam path to a target spot located inside a subject of interest;
a magnetic resonance imaging device for acquiring a magnetic resonance image of an imaging volume including the target spot;
a signal detector for acquiring a signal generated by the hadron beam; and
a controller configured to compute a position of a Bragg peak of the hadron beam based on the signal and place a representation of the position of the Bragg peak on the magnetic resonance image.

17. The medical apparatus of claim 16, wherein the signal detector is a prompt-γ system.

18. The medical apparatus of claim 16, wherein the signal detector is least one of a PET system and an ultrasound system.

19. The medical apparatus of claim 16, further comprising:
a display,
wherein the controller is further configured to represent, on a same coordinate scale, the magnetic resonance image and the position of the Bragg peak based on the acquired signal.

20. The medical apparatus of claim 16, wherein the controller is further configured to compare the position of the Bragg peak and a position of the target spot.

21. The medical apparatus of claim 20, wherein the controller is further configured to, when the position of the Bragg peak and the position of the target spot are offset by a distance greater than a tolerance, compute water equivalent path lengths of each tissue crossed by the beam path between an outer surface and the target spot based on the acquired signal.

22. The medical apparatus of claim 21, wherein the tolerance is ±10 mm or less.

23. The medical apparatus of claim 22, wherein the tolerance is ±5 mm or less.

24. The medical apparatus of claim 21, wherein the controller is further configured to optimize a treatment plan by correcting the initial beam energy such that the position of the Bragg peak and the position of the target spot are offset by a distance less than the tolerance.

25. The medical apparatus of claim 16, further comprising a hadron radiography system.

26. The medical apparatus of claim 16, further comprising a support for supporting a patient in a non-supine position.

27. A method for locating a Bragg peak of a hadron beam having a beam energy and emitted along a beam path to a target spot within a target tissue, the method comprising:
performing a magnetic resonance imaging of an imaging volume including the target spot;
acquiring a magnetic resonance image from the magnetic resonance imaging;
emitting, along the beam path to the target spot, the hadron beam having the beam energy;
detecting a signal generated by the hadron beam using a signal detector;
determining a position of the Bragg peak of the hadron beam based on the acquired signal; and
locating the Bragg peak on the magnetic resonance image.

28. The method of claim 27, further comprising representing, on a same coordinate scale, the magnetic resonance image and the position of the Bragg peak based on the acquired signal.

29. The method of claim 27, wherein the signal detector is a prom pt-γ system.

30. The method of claim 27, wherein the signal detector is least one of a PET system and an ultrasound system.

31. The method of claim 27, further comprising comparing the position of the Bragg peak and a position of the target spot.

32. The method of claim 31, further comprising, when the position of the Bragg peak and the position of the target spot are offset by a distance greater than a tolerance, compute water equivalent path lengths of each tissue crossed by the beam path between an outer surface and the target spot based on the acquired signal.

33. The method of claim 32, wherein the tolerance is ±10 mm or less.

34. The method of claim 32, wherein the tolerance is ±5 mm or less.

35. The method of claim 32, further comprising optimizing a treatment plan by correcting the beam energy such that the position of the Bragg peak and the position of the target spot are offset by a distance less than the tolerance.
